(19) United States
(12) Patent Application Publication
(10) Pub. No.: US 2023/0126803 A1
(43) Pub. Date: Apr. 27, 2023

(54) METHOD FOR CONTROLLING A LASER DEVICE FOR A LASER-INDUCED REFRACTIVE INDEX CHANGE OF A POLYMER STRUCTURE

(71) Applicant: SCHWIND EYE-TECH-SOLUTIONS GMBH, Kleinostheim (DE)

(72) Inventor: Pascal NAUBEREIT, Aschaffenburg (DE)

(21) Appl. No.: 17/951,325

(22) Filed: Sep. 23, 2022

(30) Foreign Application Priority Data
Oct. 21, 2021 (DE) 10 2021 127 400.1

(51) Int. Cl. B29D 11/00; C08J 7/12; A61F 9/008; H01S 3/10

(52) U.S. Cl. CPC B29D 11/00269; C08J 7/123; A61F 9/008; H01S 3/10038; A61F 2009/00842; A61F 2009/00872; G02B 1/041

(57) ABSTRACT

A method is disclosed for controlling a laser device for a laser-induced refractive index change (URIC) of a polymer structure. The laser device is controlled by a control device such that it emits pulsed laser pulses in a shot sequence in a preset pattern into the polymer structure. The laser pulses are emitted with preset irradiation parameters for refractive index change of the polymer structure, wherein for adjusting an order of magnitude of the refractive index change, a spatial pulse distance of the laser pulses in the polymer structure is adapted and the further irradiation parameters are kept within respective preset irradiation parameter ranges.

### FIELD

The invention relates to a method for controlling a laser device for a laser-induced refractive index change of a polymer structure, to a laser device with a control device, which is formed to perform the method, as well as to a computer program and to a computer-readable medium.

### BACKGROUND

Methods for laser-induced refractive index changes (URIC), in which the polymer structure of an artificial or biological tissue is changed by means of laser irradiation such that a phase change of the passing light and thus a refractive index change for correcting visual disorders can be achieved, are known. This treatment can also be referred to as non-surgical since a macroscopic shape of the polymer structure, for example of a cornea or an artificial lens, is not changed. This means, a lenticule is not cut out of the polymer structure to achieve a refractive power change. Herein, an adjustment of an order of magnitude of the respectively desired refractive index change is usually achieved by variation of a laser power or a laser pulse energy. Thereto, it is important that the pulse energy or average power is exactly matched and additionally fast enough to follow the very fast scanning movements. This often proves very difficult especially if the pulse energy or average power herein also has to be exactly controlled or stabilized.

### SUMMARY

The invention is based on the object to improve the control of a laser device for a laser-induced refractive index change.

This object is solved by the method according to the invention, the devices according to the invention and the computer program according to the invention. Advantageous developments are specified by the dependent claims, the description and the figures.

The invention is based on the idea that a required change of the magnitude or order of magnitude of the URIC effect within a pattern, for example radial with rotationally symmetric patterns, is not achieved via adaptation of the laser energy or laser power as usual up to now, but instead via a constant laser energy, wherein the changes of the LIRIC effect are achieved by adaptation of the spatial pulse distances.

A first aspect of the invention relates to a method for controlling a laser device for a laser-induced refractive index change (URIC) of a polymer structure. The method includes controlling the laser device by means of a control device such that it emits pulsed laser pulses in a shot sequence in a predetermined pattern into the polymer structure, wherein the laser pulses are emitted with preset irradiation parameters for refractive index change of the polymer structure, wherein a spatial pulse distance of the laser pulses in the polymer structure is adapted for adjusting an order of magnitude of the refractive index change and the further irradiation parameters are kept within respective preset irradiation parameter ranges.

By a control device, an appliance, an appliance component or an appliance group can be understood, which is configured and arranged for receiving and evaluating signals as well as for generating control signals. For example, the control device can be configured as a control appliance or control chip or computer program. For example, the control device can comprise control data in that a preset pattern is provided, which is generated in the polymer structure by means of laser-induced refractive index change. By the preset pattern, a lens, in particular a Fresnel lens, can for example be generated in the polymer structure. A polymer structure can be an artificial and/or biological polymer structure, for example a cornea of a human or animal eye. In order to obtain the effect of the refractive index change, irradiation parameters are preset, such as for example a numerical aperture, a wavelength, a temporal pulse length, a spatial pulse length, a spatial pulse distance and/or an energy, by means of which the polymer structure is to be irradiated to obtain the refractive index change. Therein, the irradiation parameters are preferably selected such that an optical breakthrough in the polymer structure, that is a generation of cavitation bubbles, is avoided.

For adjusting the order of magnitude of the refractive index change, the spatial pulse distance of the laser pulses is adjusted corresponding to the desired order of magnitude. By the order of magnitude of the refractive index change, an amount of the phase change is meant, which an area or an irradiation position of the polymer structure is to have after the irradiation. Thus, according to variation of the irradiation parameters, a higher or lower phase change and thereby refractive index change can be generated for a respective area of the polymer structure. Presently, this is achieved based on the adaptation of the spatial pulse distance, which can be faster adapted than an energy of the laser device. Thus, if a higher refractive index change for example is to be achieved, a spatial pulse distance can be decreased to generate it in an area of the polymer structure. That means, a laser pulse density can be adjusted by the variation of the spatial pulse distance. The further irradiation parameters, in particular the energy of the laser device, can be kept within a preset irradiation parameter range and preferably be constant during the adaptation of the spatial pulse distance.

By this aspect of the invention, the advantage arises that the order of magnitude of the refractive index change or the URIC effect can be fast adapted, in particular without expensively controlling the further irradiation parameters, in particular the energy. The spatial pulse distances can be adapted in relatively simple manner, for example via a pulse picker, in particular via a scanning speed and/or repetition frequency or for example via a variable path distance. Furthermore, the laser-induced refractive index change can thus be faster performed.

The invention also includes embodiments, by which additional advantages arise.

In one embodiment, it is provided that the respective order of magnitude of the refractive index change is preset for respective irradiation positions of the polymer structure, wherein the spatial pulse distances are provided depending on the respective irradiation position in the preset pattern. In other words, it can be provided in the preset pattern, in which irradiation positions of the polymer structure a larger or lower spatial pulse distance of the laser pulses is to be generated. Thus, the order of magnitude of the refractive index change can be suitably adjusted in each irradiation position. Furthermore, a lens can thus be incorporated in the polymer structure by means of the pattern, which for example corrects a visual disorder.

A further embodiment provides that the further irradiation parameters are a numerical aperture, a temporal pulse length, an energy and a wavelength. Thus, besides the spatial pulse distances, it can at least be provided that a numerical aperture of the laser device, an energy or power of the laser device, a temporal pulse length of the laser pulses and a wavelength of the laser pulses are preset, by means of which the laser-induced refractive index change can be achieved, wherein the previously mentioned irradiation parameters are preferably kept in preset irradiation parameter ranges, while the spatial pulse distances are varied.

A further advantageous embodiment provides that the spatial pulse distance is varied within a preset pulse distance range of values depending on the order of magnitude of the refractive index change to be achieved, and the further irradiation parameters are kept constant at a respective value within the respectively preset irradiation parameter ranges. In other words, the spatial pulse distance can preferably be changed within a defined range to obtain the order of magnitude of the refractive index change. The further irradiation parameters, such as for example the numerical aperture, the temporal pulse length, the energy and the wavelength, can be kept constant, preferably at a value, which is within a respectively preset irradiation parameter range, wherein the respective irradiation parameter ranges can be preset for an optimum URIC treatment. By optimum, it is meant that a URIC effect can be maximized without generating an optical breakthrough in the polymer structure. By this embodiment, the advantage arises that only the spatial pulse distance is changed and the further irradiation parameter ranges remain the same, which results in a simplification and acceleration of the irradiation of the polymer structure.

A further embodiment provides that the spatial pulse distance is changed along a scanning direction within a range between 1 nanometer and 10 micrometers, in particular between 10 nanometers and 1 micrometer, for adjusting the order of magnitude of the refractive index change. In other words, a pulse distance range of values in scanning direction can be between one nanometer and ten micrometers. The spatial pulse distance can preferably be adjusted with a suitable adjustment of the repetition frequency, which can in particular be varied between 10 kilohertz and 100 megahertz, preferably between 100 kilohertz and 100 megahertz, and/or a scanning speed. In these ranges, a laser-induced refractive index change can preferably be achieved without generating an optical breakthrough in the polymer structure.

In a further advantageous embodiment, it is provided that the spatial pulse distance is changed between adjacent laser pulse paths within a range between 10 nanometers and 50 micrometers, in particular between 50 nanometers and 5 micrometers, for adjusting the order of magnitude of the refractive index change. In these ranges too, a laser-induced refractive index change can preferably be achieved without generating an optical breakthrough in the polymer structure.

A further embodiment provides that the irradiation parameter range of a numerical aperture between 0.1 and 0.7, in particular between 0.15 and 0.35, of a temporal pulse length between 10 femtoseconds and 1 picosecond, in particular between 30 femtoseconds and 75 femtoseconds, of an energy between 1 nanojoule and 120 nanojoules, in particular between 20 nanojoules and 80 nanojoules, and of a wavelength between 300 nanometers and 1,500 nanometers, in particular between 900 nanometers and 1,000 nanometers, is preset. In other words, the further irradiation parameters can be changed within the specified ranges or preferably take a value within the specified irradiation parameter ranges, which is kept constant. Particularly preferably, the irradiation parameter ranges of the numerical aperture are between 0.15 and 0.35, of the temporal pulse length are between 30 femtoseconds and 75 femtoseconds, of the energy are between 20 nanojoules and 80 nanojoules and of the wavelength are between 900 nanometers and 1,100 nanometers, thus in an infrared wavelength range, since an optimum refractive index change can be generated with these irradiation parameter ranges without generating an optical breakthrough. In particular, the preferred irradiation parameter ranges can be ascertained from an irradiation model, which describes the refractive index change while avoiding an optical breakthrough.

A further embodiment provides that the laser pulses are emitted by a solid-state laser of the laser device, in particular a fiber laser or crystal laser. In a solid-state laser, a crystal or a glass is doped with ions, wherein these ions provide the active medium of the solid-state laser. By optical excitation of these ions, laser radiation can then be generated, for example by diode-pumped solid-state lasers. An example for a crystal laser is a so-called yttrium-aluminum-garnet laser (YAG laser), wherein these lasers can be very expensive. Therefore, fiber lasers are preferred. By a fiber laser, an appliance, an appliance group or appliance component is understood, which can comprise a fiber oscillator and/or a fiber amplifier. A fiber laser combines many advantages of individual laser types without having the corresponding disadvantages, wherefore the use of a fiber laser for laser-induced refractive index changes involves considerable advantages. A fiber laser offers a required flexibility with respect to the irradiation parameters, in particular to generate variable repetition frequencies and variable/short pulse durations, it has a required stability of the irradiation parameters, in particular an energy, pulse duration, repetition frequency and pulse shape, and has an increased freedom from maintenance. In particular, many irradiation parameters can be easier achieved with a fiber laser. By this embodiment, the advantage arises that the control of the laser device can be more accurately adjusted.

A further embodiment provides that the laser pulses are emitted into a biopolymer, in particular a cornea of a human or animal eye. In other words, the polymer structure is a biopolymer, in which the preset pattern with the refractive index change adjusted by the spatial pulse distance is generated. Hereby, the advantage arises that a treatment of a human or animal eye can be improved.

In a further embodiment, it is provided that the laser pulses are emitted into a plastic polymer, in particular for generating an artificial lens. In other words, the polymer structure is a plastic polymer. By this embodiment, artificial lenses can in particular be generated in improved manner.

A further advantageous embodiment provides that the spatial pulse distance is adjusted by a pulse picker of the laser device and/or a scanning speed and/or a pulse path distance of adjacent laser pulse paths. A pulse picker is an electrically controllable optical switch, which can discard individual laser pulses from a pulse train and thus only passes the desired number of the pulses. Hereto, an acousto-optical pulse picker can in particular be used. Alternatively or additionally, the scanning speed can be varied across the polymer structure and/or a pulse path distance of adjacent laser pulse paths can be suitably preset. By this form of configuration, one obtains a preferred possibility of adjustment for the spatial pulse distance.

A further advantageous embodiment provides that a Fresnel lens is generated in the polymer structure as the preset pattern. A Fresnel lens is a stepped lens, which comprises prismatic partial sections, which are in particular constructed as a parallel strip or concentric circular rings.

A second aspect of the present invention relates to a laser device with at least one control device. The control device can be configured to perform one of the above described embodiments of the method according to the invention. The above cited advantages arise. The control device can for example be configured as a control chip, control appliance or application program (“app”). The control device can preferably comprise a processor device and/or a data storage. By a processor device, an appliance or an appliance component for electronic data processing is understood. The processor device can for example comprise at least one microcontroller and/or at least one microprocessor. Preferably, a program code for performing the method according to the invention can be stored on the optional data storage. The program code can then be configured, upon execution by the processor device, to cause the control device to perform one of the above described embodiments of the method according to the invention. Furthermore, the laser device can comprise a laser, which is formed to generate pulsed laser pulses, in particular for the laser-induced refractive index change in the polymer structure. The above described advantages arise.

In an advantageous configuration of the laser device, it is provided that the laser device comprises a solid-state laser, in particular a fiber laser. By a fiber laser, an appliance, an appliance group or appliance component is understood, which includes a fiber oscillator and/or a fiber amplifier. A fiber laser combines many advantages of the individual laser types without having the corresponding disadvantages, wherefore the use of a fiber laser for the laser-induced refractive index change of a polymer structure involves considerable advantages. A fiber laser offers the required flexibility with respect to the parameter space (in particular for example variable repetition rate and variable/short pulse duration), the required stability of the parameters (in particular for example pulse energy, pulse duration, repetition rate and pulse shape) and an increased freedom from maintenance (for example an air cooling (“air-cooled”) and a long lifetime). The flexibility with respect to the parameter space arises in that many parameters can be easier achieved with a fiber laser. Therein, a fiber oscillator and a fiber amplifier can for example be encompassed by the fiber laser according to the invention, but for example also a fiber oscillator and a solid-state amplifier. By the employment of the fiber laser, which is only used for removing lenticules in the prior art up to now, thus, for surgical and thereby invasive methods, a non-surgical or incision-free method is optimized by the invention.

In a further advantageous configuration of the laser device according to the invention, the laser device can be suitable to emit laser pulses in a wavelength range between 300 nm and 1500 nm, preferably between 900 nm and 1100 nm, at a respective pulse duration between 10 fs and 1 ps, preferably between 30 fs and 75 fs, and a repetition frequency of greater than 10 kilohertz (kHz), preferably between 100 kHz and 100 megahertz (MHz). The already above mentioned advantages arise.

In further advantageous configurations of the laser device according to the invention, the control device can comprise at least one storage device for at least temporary storage of at least one control dataset, wherein the control dataset or datasets include(s) control data for positioning and/or for focusing and/or for irradiation parameter adjustment of individual laser pulses; and can comprise at least one beam device for beam guidance and/or beam shaping and/or beam deflection and/or beam focusing of a laser beam of the laser. Therein, the mentioned control datasets are preferably generated based on a measured topography and/or pachymetry and/or morphology.

Further features and the advantages thereof can be taken from the descriptions of the first inventive aspect, wherein advantageous configurations of each inventive aspect are to be regarded as advantageous configurations of the respectively other inventive aspect.

A third aspect of the invention relates to a computer program including commands, which cause the laser device according to the second inventive aspect to execute the method steps according to the first inventive aspect.

A fourth aspect of the invention relates to a computer-readable medium, on which the computer program according to the third inventive aspect is stored. Further features and the advantages thereof can be taken from the descriptions of the first to third inventive aspects, wherein advantageous configurations of each inventive aspect are to be regarded as advantageous configurations of the respectively other inventive aspect.

### BRIEF DESCRIPTION OF THE DRAWINGS

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 depicts a schematic representation of a laser device according to an exemplary embodiment.

In the figures, identical or functionally identical elements are provided with the same reference characters.

### DETAILED DESCRIPTION

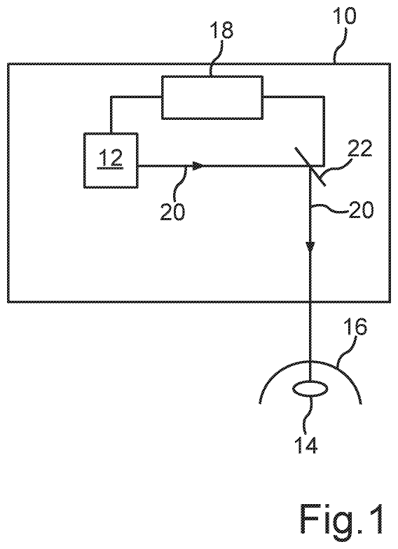

FIG. 1 shows a schematic representation of a laser device 10 with a laser 12, in particular a solid-state laser, for the laser-induced refractive index change (URIC) of a polymer structure 14. In this embodiment, the polymer structure 14 can be a biopolymer, in particular an area of a cornea 14 of a human or animal eye 16. Alternatively, the polymer structure can also be a plastic polymer, into which an artificial lens is incorporated. A laser pulse sequence, a laser pulse distribution and irradiation parameters for the refractive index change of the cornea 14 can be preset in the form of control data by a control device 18, such that the laser 12 emits pulsed laser pulses to laser pulse positions preset by the control data with irradiation parameters provided by the control data to achieve the refractive index change. In particular, a pattern can be preset, which is to be generated in the cornea 14. Alternatively, the control device 18 can be a control device 18 external with respect to the laser device 10.

Furthermore, FIG. 1 shows that the laser beam 20 generated by the laser 12 can be deflected towards the eye 16 by means of a beam deflection device 22, namely a beam deflection apparatus such as for example a rotation scanner, to generate the refractive index change in the cornea 14 according to the preset pattern. The beam deflection apparatus 22 can also be controlled by the control device 18.

Preferably, the illustrated laser 12 can be a fiber laser, which is at least formed to emit laser pulses in a wavelength range between 800 nanometers and 1500 nanometers, preferably between 900 nanometers and 1100 nanometers, at a respective pulse duration between 10 femtoseconds and 90 femtoseconds, preferably between 30 femtoseconds and 75 femtoseconds, and a repetition frequency of greater than 10 kilohertz, preferably between 100 kilohertz and 100 megahertz.

Optionally, the control device 18 additionally comprises a storage device (not illustrated) for at least temporary storage of at least one control dataset, wherein the control dataset or datasets include(s) control data for irradiation parameter adjustment, positioning and/or focusing of individual laser pulses in the eye 16.

For adjusting an order of magnitude of the refractive index change in the cornea 14, it can be provided that the spatial pulse distances are varied depending on the respective irradiation position to for example generate a Fresnel lens in the cornea 14. With a great refractive index change to be achieved, a spatial pulse distance can thus for example be reduced, and with a small refractive index change to be achieved, a spatial pulse distance can be increased. Thereto, the spatial pulse distance can be varied within a preset pulse distance range of values, which can take values between 1 nanometer and 10 micrometers along a scanning direction and a value between 10 nanometers and 50 micrometers between adjacent laser pulse paths. In order to adjust the spatial pulse distance, the laser device 10 can comprise a pulse picker (not shown), by which individual laser pulses can be excluded from a laser pulse train, such that a larger pulse distance arises in particular with consistent scanning speed by the beam deflection device 22. Alternatively or additionally, the scanning speed of the beam deflection device 22 can be adapted to vary the spatial pulse distance, and/or a pulse path distance of adjacent laser pulse paths can be adjusted by the beam deflection device 22 to control the order of magnitude of the refractive index change.

The further irradiation parameters, which are preset for the laser-induced refractive index change, such as for example the numerical aperture, a temporal pulse length, an energy and a wavelength, can be kept within preset irradiation parameter ranges, preferably at a constant value within the preset irradiation parameter ranges. Therein, the irradiation parameter range of the numerical aperture can be between 0.1 and 0.7, preferably between 0.15 and 0.35, an irradiation parameter range of a temporal pulse length can be between 10 femtoseconds and 1 picosecond, preferably between 30 femtoseconds and 75 femtoseconds, an irradiation parameter range of an energy can be between 1 nanojoule and 120 nanojoules, preferably between 20 nanojoules and 80 nanojoules, and an irradiation parameter range of a wavelength can be between 300 nanometers and 1,500 nanometers, preferably between 900 nanometers and 1,100 nanometers.

Overall, the examples show how a change of the magnitude of the URIC effect can be adapted within a preset pattern by the invention without varying an energy of the laser device.

1. A method for controlling a laser device for a laser-induced refractive index change (URIC) of a polymer structure, comprising:
controlling the laser device using a control device such that the laser device emits pulsed laser pulses in a shot sequence in a preset pattern into the polymer structure,
wherein the laser pulses are emitted with preset irradiation parameters for refractive index change of the polymer structure, and
wherein for adjusting an order of magnitude of the refractive index change, a spatial pulse distance of the laser pulses in the polymer structure is adapted and the further irradiation parameters are kept within respective preset irradiation parameter ranges.

2. The method according to claim 1, wherein the respective order of magnitude of the refractive index change is preset for respective irradiation positions of the polymer structure, and wherein the spatial pulse distances are provided depending on the respective irradiation position in the preset pattern.

3. The method according to claim 1, wherein the further irradiation parameters are a numerical aperture, a temporal pulse length, an energy and a wavelength.

4. The method according to claim 1, wherein the spatial pulse distance is changed within a preset pulse distance range of values depending on the order of magnitude of the refractive index change to be achieved and the further irradiation parameters are kept constant at a respective value within the respectively preset irradiation parameter ranges.

5. The method according to claim 1, wherein the spatial pulse distance is varied along a scanning direction within a range between 1 nm and 10 μm, in particular between 10 nm and 1 μm, for adjusting the order of magnitude of the refractive index change.

6. The method according to claim 1, wherein the spatial pulse distance is changed between adjacent laser pulse paths within a range between 10 nm and 50 μm, in particular between 50 nm and 5 μm, for adjusting the order of magnitude of the refractive index change.

7. The method according to claim 1, wherein the irradiation parameter range of a numerical aperture between 0.1 and 0.7, in particular between 0.15 and 0.35, of a temporal pulse length between 10 fs and 1 ps, in particular between 30 fs and 75 fs, of an energy between 1 nJ and 120 nJ, in particular between 20 nJ and 80 nJ, and of a wavelength between 300 nm and 1500 nm, in particular between 900 nm and 1100 nm, is preset.

8. The method according to claim 1, wherein the laser pulses are emitted by a solid-state laser of the laser device, in particular a fiber laser or crystal laser.

9. The method according to claim 1, wherein the laser pulses are emitted into a biopolymer, in particular a cornea of a human or animal eye.

10. The method according to claim 1, wherein the laser pulses are emitted into a plastic polymer, in particular for generating an artificial lens.

11. The method according to claim 1, wherein the spatial pulse distance is adjusted by a pulse picker of the laser device and/or a scanning speed and/or a pulse path distance of adjacent laser pulse paths.

12. The method according to claim 1, wherein a Fresnel lens is generated in the polymer structure as the preset pattern.

13. A laser device with a control device, which is configured to perform a method according to claim 1.

14. The laser device according to claim 13, wherein the laser device comprises a solid-state laser, in particular a fiber laser.

15. The laser device according to claim 13, wherein the laser device is suitable to emit laser pulses in a wavelength range between 300 nm and 1500 nm, preferably between 900 nm and 1100 nm, at a respective pulse duration between 10 fs and 1 ps, preferably between 30 fs and 75 fs, and a repetition frequency of greater than 10 kHz, preferably between 100 kHz and 100 MHz.

16. The laser device according to claim 13, wherein the control device:
comprises at least one storage device for at least temporary storage of at least one control dataset, wherein the control dataset or datasets include(s) control data for positioning and/or for focusing and/or for irradiation parameter adjustment of individual laser pulses; and
includes at least one beam device for beam guidance and/or beam shaping and/or beam deflection and/or beam focusing of a laser beam of the laser device.

17. A computer program including commands, which cause a laser device with a control device to execute a method according to claim 1.

18. A non-transitory computer-readable medium, on which the computer program according to claim 17 is stored.
